(19) United States
(12) Patent Application Publication
(10) Pub. No.: US 2026/0047786 A1
(43) Pub. Date: Feb. 19, 2026

(54) Syringe With Integrated Test Strip

(71) Applicant: Zachary Hagen, Baltimore, MD (US)

(72) Inventor: Zachary Hagen, Baltimore, MD (US)

(21) Appl. No.: 18/804,385

(22) Filed: Aug. 14, 2024

(51) Int. Cl. A61B 5/157; A61B 5/00; A61B 5/15; A61B 5/153

(52) U.S. Cl. CPC A61B 5/157; A61B 5/150236; A61B 5/153; A61B 5/4845; A61B 2562/0295

(57) ABSTRACT

A syringe includes a barrel, a plunger configured to slidingly engage the barrel to receive or dispense a liquid, and a test strip disposed on or in the barrel and configured to detect for a presence of one or more substances in the liquid.

### TECHNICAL FIELD

This application relates generally to syringes.

### BACKGROUND

Recreational drugs, such as opioids, can be delivered through intravenous injection using syringes. Recently, recreational drugs have been laced or contaminated with more-powerful drugs such as fentanyl, xylazine, and medetomidine. This has led to an increase in drug overdoses since the end-user usually does not know whether the recreational drug includes fentanyl or other adulterants. Harm reduction campaigns often distribute clean needles and fentanyl test strips as separate products, both of which have been shown to reduce the mortality of illicit drug use. Discussions with people who use illicit drugs have revealed that a large barrier to the use of fentanyl test strips is the fact that part of the user's product must be put aside for testing, making it unusable.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages, and novel features of the disclosure will be set forth in the following detailed description of the disclosure when considered in conjunction with the drawings, which are intended to illustrate, not limit, the invention.

An aspect of the invention is directed to a syringe comprising a barrel; a plunger configured to slidingly engage the barrel to receive or dispense a liquid; and a test strip disposed on or in the barrel and configured to detect for a presence of one or more substances in the liquid.

In one or more embodiments, the one or more substances comprise an opioid. In one or more embodiments, the opioid comprises fentanyl. In one or more embodiments, the plunger is configured to crush the test strip when the plunger is moved towards a tip of the barrel.

In one or more embodiments, the syringe further comprises a covering material attached to an internal wall of the barrel, the test strip disposed between the covering material and the internal wall, wherein a test window is defined in the covering material at a base of the test strip, and only the base of the test strip is configured to be exposed to the liquid. In one or more embodiments, a test region of the test strip faces the internal wall. In one or more embodiments, the covering material comprises a tube. In one or more embodiments, the covering material comprises a plastic and/or a polymer.

In one or more embodiments, the syringe further comprises a covering material attached to an internal wall of the barrel, the test strip disposed between the covering material and the internal wall, wherein a channel is defined in the internal wall, the channel extending to a base of the test strip to form a test window, and only the base of the test strip is configured to be exposed to the liquid. In one or more embodiments, the test strip is disposed in a cavity defined in an internal wall of the barrel, and a channel is defined between the cavity and an internal volume of the barrel, the channel aligned with a base of the test strip such that only the base of the test strip is configured to be exposed to the liquid.

Another aspect of the invention is directed to a syringe comprising a barrel defining a test window; a plunger configured to slidingly engage the barrel to receive or dispense a liquid; and a test strip configured to detect for a presence of one or more substances in the liquid, the test strip disposed on an external wall of the barrel, the test strip having a base in fluid communication with the test window such that only the base of the test strip is configured to be exposed to the liquid.

In one or more embodiments, the test window includes a channel that extends from an internal wall to the external wall of the barrel. In one or more embodiments, the syringe further comprises a covering material attached to the external wall, the test strip disposed between the covering material and the external wall. In one or more embodiments, the covering material comprises a shrink wrap. In one or more embodiments, the covering material comprises a tube In one or more embodiments, the test strip is encased in the covering material.

Another aspect of the invention is directed to a syringe comprising a barrel; a plunger configured to slidingly engage the barrel to receive or dispense a liquid, the plunger defining a test window and a cavity; and a test strip configured to detect for a presence of one or more substances in the liquid, the test strip disposed in the cavity, the test strip having a base in fluid communication with the test window such that only the base of the test strip is configured to be exposed to the liquid.

In one or more embodiments, the test window is defined in a base of the plunger. In one or more embodiments, portions of the test strip other than the base are fluidly sealed in the cavity.

### BRIEF DESCRIPTION OF THE DRAWINGS

For a fuller understanding of the nature and advantages of the concepts disclosed herein, reference is made to the detailed description of preferred embodiments and the accompanying drawings.

FIG. 1 is a side view of a syringe according to an embodiment.

FIG. 2 is a side view of an internal wall of the barrel illustrated in FIG. 1, as seen from inside of the barrel, according to an embodiment.

FIG. 3 is a side view of an internal wall of the barrel illustrated in FIG. 1, as seen from inside of the barrel, according to another embodiment.

FIG. 4 is a cross section of the barrel taken through a first plane in FIG. 2 according to different embodiments.

FIG. 5 is a cross section of the barrel taken through plane a second plane in FIG. 2 according to different embodiments.

FIG. 6 is a cross section of the barrel taken through the second plane in FIG. 2 according to different embodiments.

FIGS. 7A and 7B are cross sections of the barrel taken through a first plane in FIG. 3 according to different embodiments.

FIGS. 8A and 8B are cross sections of the barrel taken through a second plane in FIG. 3 according to different embodiments.

FIGS. 9A and 9B are cross sections of the barrel taken through the second plane in FIG. 3 according to different embodiments.

FIGS. 10A and 10B are cross sections of the syringe illustrated in FIG. 1 according to different embodiments.

FIG. 11 is a side view of a syringe when the plunger is pushed forward according to an embodiment.

FIG. 12 is a side view of an internal wall of the barrel illustrated in FIG. 1, as seen from inside of the barrel, according to another embodiment.

FIG. 13 is a cross section of the internal wall of the barrel illustrated in FIG. 12 according to an embodiment.

FIG. 14 is a side view of an external wall of a barrel, as seen from the outside of the barrel, according to an embodiment.

FIG. 15 which is a cross section of the external wall of the barrel illustrated in FIG. 14 according to an embodiment.

FIG. 16 is a side view of an external wall of a barrel, as seen from the outside of the barrel, according to another embodiment.

FIG. 17 is a cross section of the external wall of the barrel illustrated in FIG. 16 according to an embodiment.

FIGS. 18A and 18B are cross sections of the barrel illustrated in FIG. 17 taken through a first plane according to different embodiments.

FIGS. 19A and 19B are cross sections of the barrel illustrated in FIG. 17 taken through a second plane according to different embodiments.

FIG. 20 is a side view of a syringe according to another embodiment.

FIG. 21 is a cross section of the plunger illustrated in FIG. 20 according to an embodiment.

### DETAILED DESCRIPTION

A syringe is integrated with a test strip that is configured to automatically test a liquid in the syringe for one or more substances. In some embodiments, the test strip is integrated on or in the barrel of the syringe. An exposure window can be defined in the barrel or in a covering material that allows the liquid to flow onto a base of the test strip to automatically perform the test as the liquid is drawn into the syringe. In other embodiments, the test strip is combined with the plunger of the syringe. An exposure window can be defined at a base of the plunger to allow the liquid to flow onto the base of the test strip to automatically perform the test.

The disclosed syringes can be distributed to users in order to increase testing for fentanyl and other adulterants prior to injection without the users having to “waste” any product for testing.

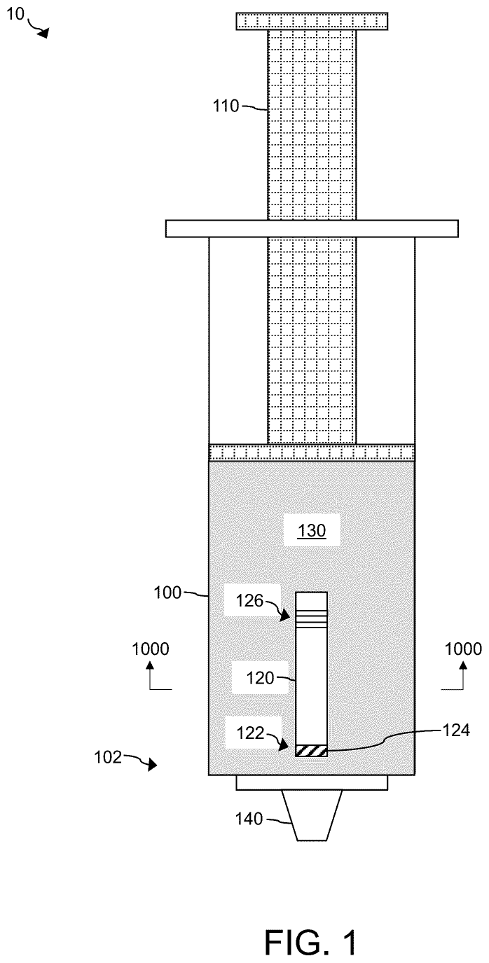

FIG. 1 is a side view of a syringe 10 according to an embodiment. The syringe 10 includes a barrel 100, a plunger 110, and a test strip 120. The plunger 110 is disposed within the barrel 100 and configured to slidingly engage the barrel 100 to receive and/or dispense a liquid 130. The liquid 130 can be or include a pharmaceutical, a recreational drug (e.g., an opioid such as heroin and/or a stimulant such as methamphetamine), or another substance. The test strip 120 can be disposed on or in the barrel 100. At least a portion of the test strip 120 is configured to be in fluid communication with the liquid 130 such that the test strip 120 can automatically test the liquid 130 for the presence of one or more substances. In one example, the test strip 120 can automatically test the liquid 130 for the presence of fentanyl or other recreational drug adulterants.

In some embodiments, only a base 122 of the test strip 120 is configured to be in fluid communication with the liquid 130. The base 122 includes a sample pad 124 such as for a lateral flow immunoassay test strip. For example, an exposure window can be formed or defined that can allow the liquid 130 to flow onto the base 122 (e.g., onto the sample pad 124) while the liquid 130 is restricted from flowing onto the rest of the test strip 120. This allows the test strip to automatically wick the fluid along the immunoassay similar to the testing process in a typical non-integrated lateral flow immunoassay. The exposure window can be formed or defined using a covering material and/or a wall of the barrel 100.

In other embodiments, all of test strip 120 can come into contact with the liquid 130 (e.g., depending on the volume of the liquid 130 in the barrel 100) for example when the test strip 120 is not a lateral-flow test strip. These contact-based test strips 120 can include or consist of litmus paper, pH strips, and/or other contact-activated strips that may be read through the clear syringe wall 100 before dispensing the fluid 130. For example, the test strip 120 can freely float in the liquid 130 in some embodiments. In other embodiments, a back of the test strip 120 can be attached (e.g., using an adhesive) to the inside wall of the barrel 100. Potential uses of this embodiment include litmus/pH testing, nitrate/nitrite testing, salinity testing, and/or ammonia testing.

The test strip 120 can be disposed at or near a distal end 102 of the barrel 100. For example, the test strip 120 can be located near a tip 140 of the syringe 10. Placing the test strip 120 near the distal end 102 of the barrel 100 can ensure that the test strip 120 performs when only a small volume of the liquid 130 is in the barrel 100. For example, when the test strip 120 is placed near the distal end 102 of the barrel 100, the test strip 120 can perform the test when the barrel 100 holds a volume of liquid 130 that corresponds to only about 5% to about 10% of the maximum total volumetric capacity of the barrel 100.

The test strip 120 can be disposed on or in the barrel 100 and can be configured and arranged such that one or more test lines 126 is/are visible through the barrel 100. Multiple test strips can also be integrated into the barrel of the syringe 100 to allow for testing of multiple substances from one syringe.

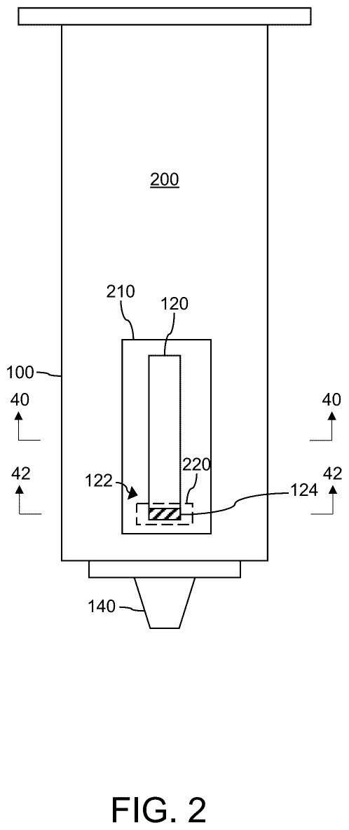

FIG. 2 is a side view of an internal wall 200 of the barrel 100, as seen from inside of the barrel 100, according to an embodiment. The plunger 110 and liquid 130 are not illustrated in this figure for clarity purposes only.

The test strip 120 is partially covered and/or encased by a covering material 210, which is shown as transparent so that the test strip 120 is viewable. The test strip 120 is oriented such that the test line(s) 126 (FIG. 1) face the transparent internal wall 200 so as to be viewable through the barrel 100 from an external vantage point.

An exposure window 220 is defined in the covering material 210. The exposure window 220 is aligned with the base 122 of the test strip 120. The exposure window 220 is configured to allow a liquid 130 to pass onto the base 122 to wet (e.g., make direct physical contact with) the sample pad 124. The covering material 210 is configured to block the rest of the test strip 120 such that the exposure window 220 is the only path for the liquid 130 to reach the test strip 120. Thus, the covering material 210 and the exposure window 220 are configured such that only the base 122 and sample pad 124 are in fluid communication with and/or in direct physical contact with the liquid 130, which is advantageous for example when the test strip 120 is a lateral flow test strip (e.g., a lateral flow immunoassay test strip). The liquid (e.g., liquid 130) can flow or pass through the exposure window 220 and onto the base 122 and/or the sample pad 124 of the test strip 120 to allow the test strip 120 to automatically test the liquid for the presence of one or more substances.

The covering material 210 can include or consist of a plastic material and/or a polymer material, such as polypropylene in a medical setting. The covering material 210 can be chemically inert to the liquid 130. The covering material 210 can be attached to the internal wall 200 using an adhesive, laser welds, and/or other plastic polymer attachment means such as polypropylene-based adhesives or inert ethyl 2-cyanoacrylate adhesives. In some embodiments, the covering material 210 is optically transparent.

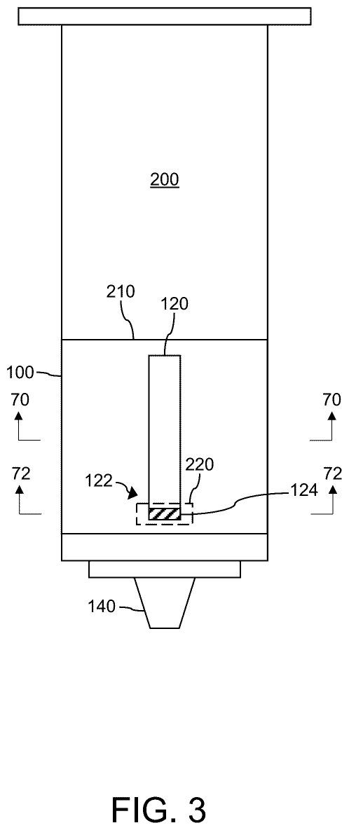

In some embodiments, the covering material 210 is disposed on and covers only the test strip 120 and the internal wall 200 in close proximity to the test strip 120, for example as illustrated in FIG. 2. In other embodiments, the covering material can extend along some or all of the circumference of the internal wall 200, for example as illustrated in FIG. 3. The circumference of the internal wall 200 can be the same as the internal circumference and/or the internal perimeter of the barrel 100 to completely encase the test strip 210 along the inner wall 100.

The test strip 120 can be oriented such that the test line(s) 126 is/are facing the transparent internal wall 200 such that the test line(s) 126 is/are viewable externally in FIG. 1 but not in FIG. 2 or 3. In these examples the sample pad 124 is present on both sides of the test strip, however the immunoassay only occurs on the side that abuts the internal syringe wall 100 in order to allow the test strip results to be viewed from the outside of the syringe.

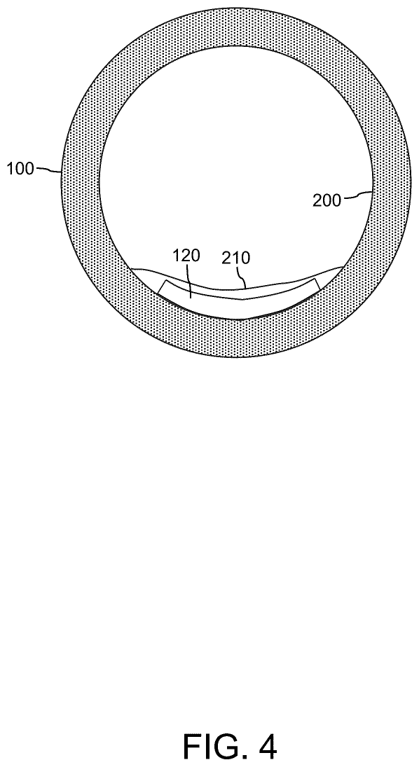

FIG. 4 is a cross section of the barrel 100 taken through plane 40 in FIG. 2 according to one or more embodiments. The covering material 210 is disposed on and covers the test strip 120. The thickness of the covering material 210 can taper in regions away from the test strip 120 to allow the plunger 110 to travel freely along the length of the barrel 100 while maintaining the suction necessary to draw up the substrate for testing. The covering material 210 can be disposed on and cover the test strip 120 and the internal wall 200 in close proximity to the test strip 120.

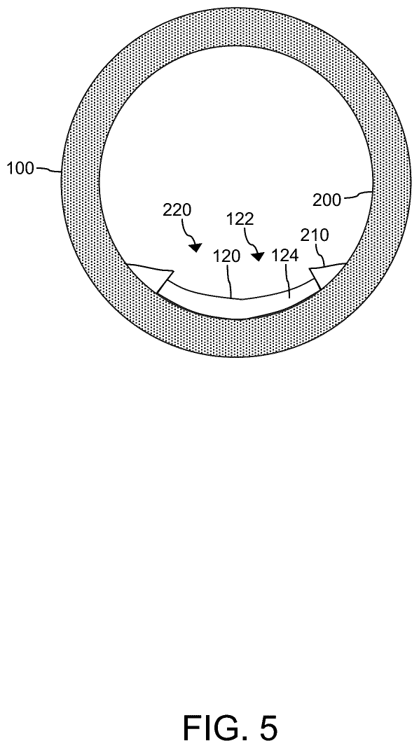

FIG. 5 is a cross section of the barrel 100 taken through plane 42 in FIG. 2 according to one or more embodiments. The covering material 210 is removed on the inside edge of the test strip 120 to form an exposure window 220. The liquid (e.g., liquid 130) can flow or pass through the exposure window 220 and onto the base 122 and/or the sample pad 124 of the test strip 120 to allow the test strip 120 to automatically test the liquid for the presence of one or more substances. The thickness of the covering material 210 can taper in regions away from the test strip 120. The embodiment shown in FIG. 4 can correspond with the embodiment shown in FIG. 5, such that FIGS. 4 and 5 can represent an implementation of FIG. 2.

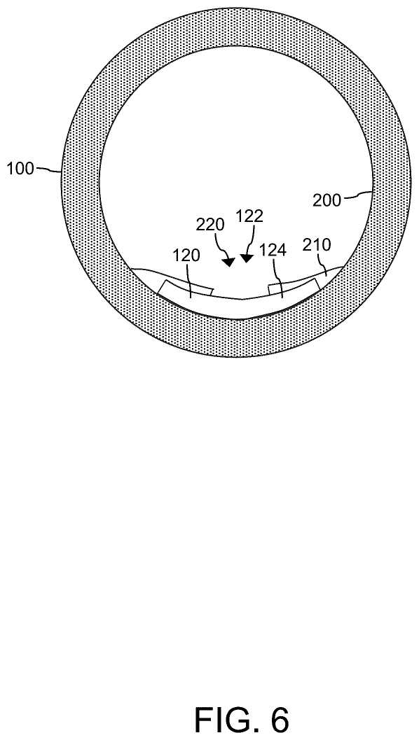

FIG. 6 is a cross section of the barrel 100 taken through plane 42 in FIG. 2 according to one or more embodiments. The covering material 210 is only partially removed on the inside edge of the test strip 120 to form an exposure window 220. The size (e.g. width) of the exposure window 220 is smaller than the corresponding size (e.g., width) of the test strip 120. The liquid (e.g., liquid 130) can flow or pass through the exposure window 220 and onto the base 122 and/or the sample pad 124 of the test strip 120 to allow the test strip 120 to automatically test the liquid for the presence of one or more substances. The thickness of the covering material 210 can taper in regions away from the test strip 120. The embodiment shown in FIG. 4 can correspond with the embodiment shown in FIG. 6, such that FIGS. 4 and 6 can represent an implementation of FIG. 2.

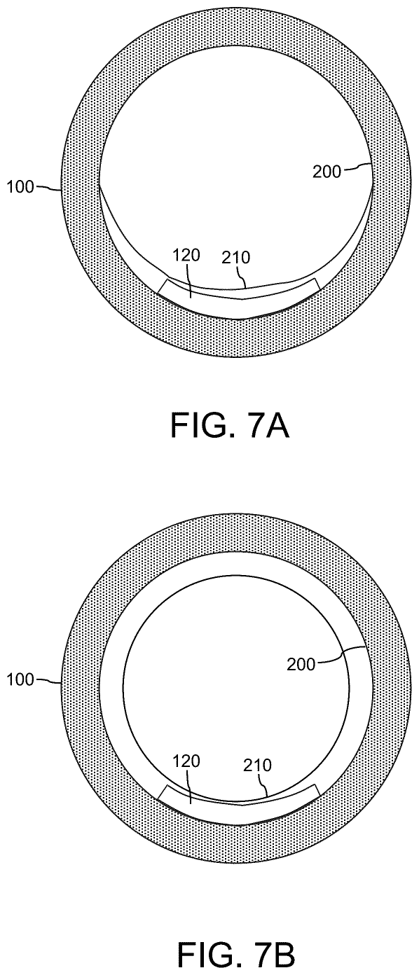

FIGS. 7A and 7B are cross sections of the barrel 100 taken through plane 70 in FIG. 3 according to different embodiments. In FIG. 7A, the covering material 210 is disposed on and covers the test strip 120 and about half of the circumference of the internal wall 200. The thickness of the covering material 210 tapers in regions away from the test strip 120. Alternatively, in FIG. 7A the thickness of the covering material 210 can be substantially uniform along the circumference of the internal wall 200 (e.g., similar to FIG. 4B). In FIG. 7B, the covering material 210 is disposed on and covers the test strip 120 and the entire circumference of the internal wall 200, such that the covering material 210 comprises a tube, a hollow cylinder, or a ring. FIGS. 7A and 7B can be alternative implementations of the embodiment illustrated in FIG. 3.

While example embodiments are illustrated, it is understood that the covering material 210 can be disposed on and cover different percentages of the circumference of the internal wall 200 other than those illustrated. For example, the covering material 210 can be disposed on and cover about 10% to about 100% of the circumference of the internal wall 200, including about 25%, about 50%, about 75%, and any range or value between any two of the foregoing percentages. The thickness of the covering material 210 can be uniform or variable along the circumference of the internal wall 200.

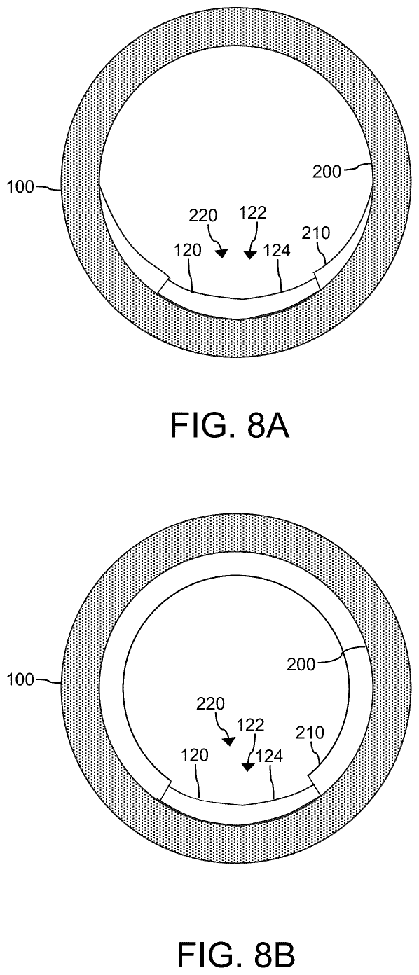

FIGS. 8A and 8B are cross sections of the barrel 100 taken through plane 72 in FIG. 3 according to different embodiments. In both embodiments, the covering material 210 is removed on the inside edge of the test strip 120 to form an exposure window 220. The liquid (e.g., liquid 130) can flow or pass through the exposure window 220 and onto the base 122 and/or the sample pad 124 of the test strip 120 to allow the test strip 120 to automatically test the liquid for the presence of one or more substances.

The embodiment shown in FIG. 8A can correspond with the embodiment shown in FIG. 7A. The embodiment shown in FIG. 8B can correspond with the embodiment shown in FIG. 7B.

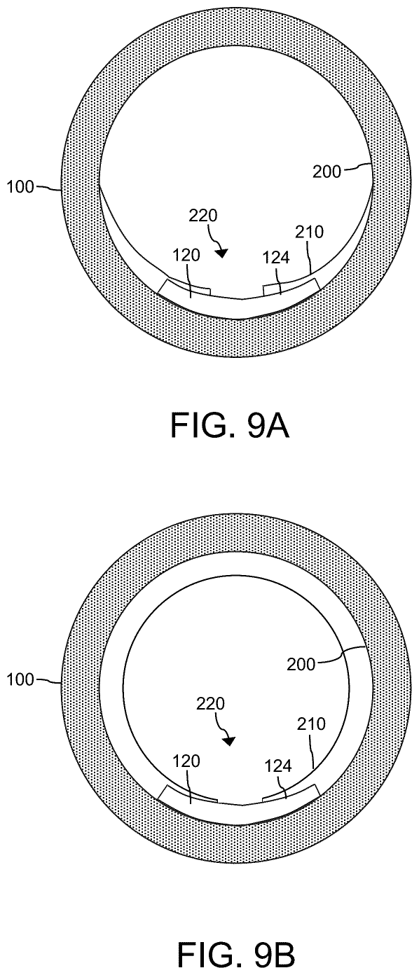

FIGS. 9A and 9B are cross sections of the barrel 100 taken through plane 72 in FIG. 3 according to different embodiments. In both embodiments, the covering material 210 is only partially removed on the inside edge of the test strip 120 to form an exposure window 220. The size (e.g. width) of the exposure window 220 is smaller than the corresponding size (e.g., width) of the test strip 120. The embodiment shown in FIG. 7A can correspond with the embodiment shown in FIG. 9A, such that FIGS. 7A and 9A can represent an implementation of FIG. 3. The embodiment shown in FIG. 7B can correspond with the embodiment shown in FIG. 9B, such that FIGS. 7B and 9B can represent another implementation of FIG. 3.

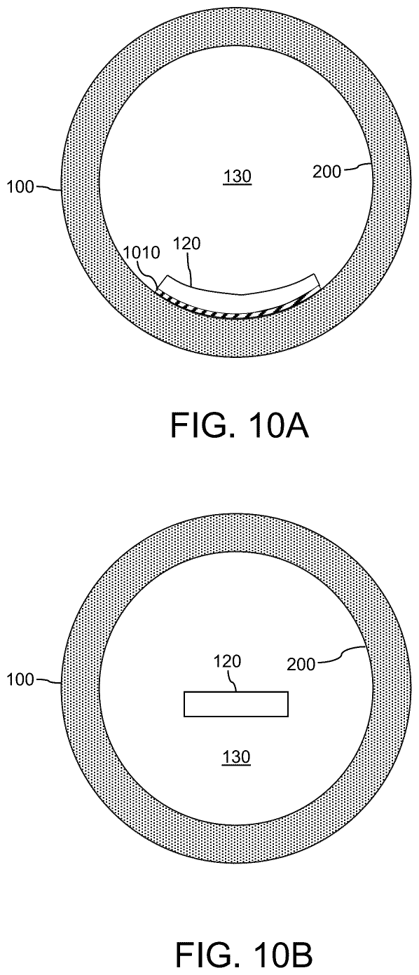

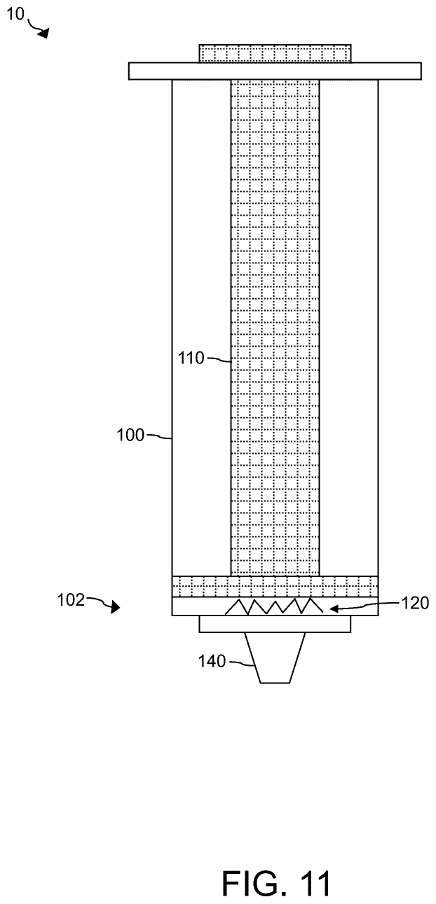

FIGS. 10A and 10B are cross sections of the syringe 10 taken through plane 1000 in FIG. 1 according to different embodiments. In FIG. 10A, the test strip 120 is attached to the internal wall 200 with an adhesive 1010. In FIG. 10B, the test strip 120 is not attached to the internal wall 200 and allowed to freely float within the liquid 130. In both FIGS. 10A and 10B, all of test strip 120 can come into contact with the liquid 130 (e.g., depending on the volume of the liquid 130 in the barrel 100), for example when the test strip 120 is not a lateral-flow test strip. The result of the test strip may be read through the transparent wall of the syringe 10. When the plunger 110 is pushed forward to dispense the liquid 130, the test strip 120 may be partially or fully crushed, for example as illustrated in FIG. 11.

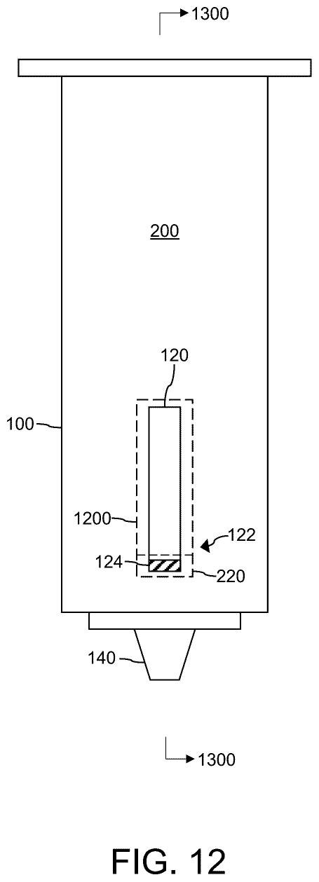

FIG. 12 is a side view of an internal wall 200 of the barrel 100, as seen from inside the barrel 100, according to another embodiment. The plunger 110 and liquid 130 are not illustrated in this figure for clarity purposes only. The test strip 120 is disposed in a cavity 1200 defined in the internal wall 200. An exposure window 220 is defined in the internal wall 200 to allow a liquid (e.g., liquid 130) to pass onto the base 122 to wet (e.g., make direct physical contact with) the sample pad 124.

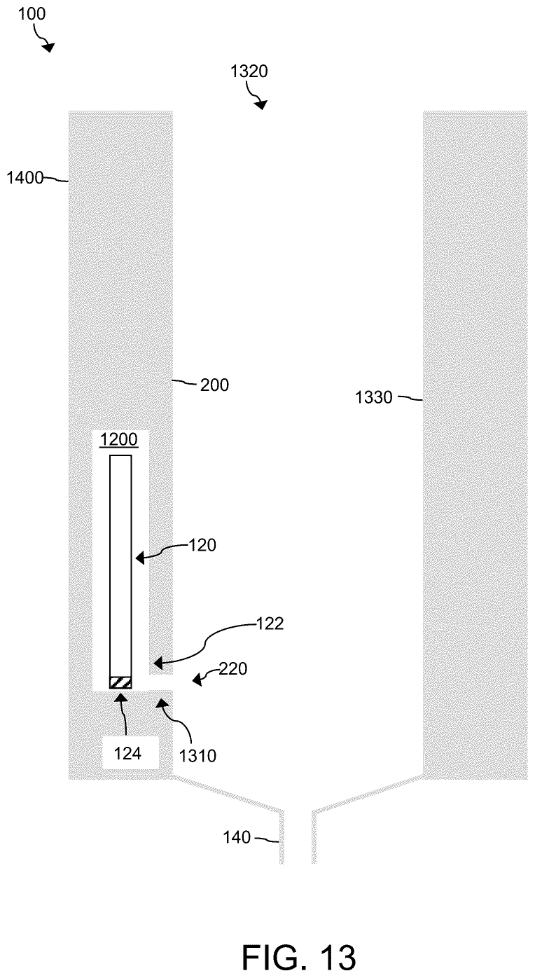

FIG. 13 is a cross section of the internal wall 200 of the barrel 100 through plane 1300 in FIG. 12 according to an embodiment. The exposure window 220 includes a channel 1310 that extends (e.g., radially extends) between an internal volume or cavity 1320 of the barrel 100, that includes the liquid (e.g., liquid 130), and the cavity 1200. A second internal wall 1330 (e.g., the other internal side of the barrel 100) and the tip 140 are shown in FIG. 13 to illustrate the internal volume/cavity 1320. The cross-sectional thickness of the tip 140 is not to scale. The test strip 120 can be oriented such that the test line(s) 126 (FIG. 1) face an external wall 1400 of the barrel 100 so as to be viewable through the barrel 100.

The channel 1310 is aligned with the base 122 and/or the sample pad 124 of the test strip 120. The liquid (e.g., liquid 130) can flow or pass through the exposure window 220 and the channel 1310 and onto the base 122 and/or the sample pad 124 of the test strip 120 to allow the test strip 120 to automatically test the liquid for the presence of one or more substances.

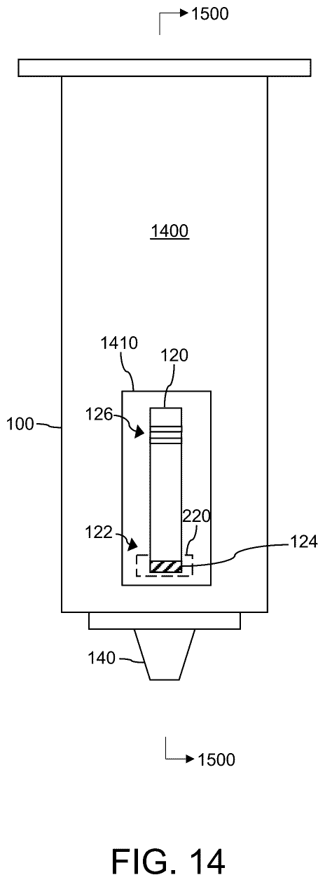

FIG. 14 is a side view of an external wall 1400 of the barrel 100, as seen from the outside of the barrel 100, according to an embodiment. The plunger 110 and liquid 130 are not illustrated in this figure for clarity purposes only.

The test strip 120 is disposed on the external wall 1400 of the barrel 100 and is partially covered and/or encased by a covering material 1410. The covering material 1410 is optically transparent so that the test strip 120 is viewable. The covering material 1410 can be the same as or different than the covering material 210. The test strip 120 can be oriented such that the test line(s) 126 (FIG. 1) face away from the external wall 1400 so as to be viewable by a user.

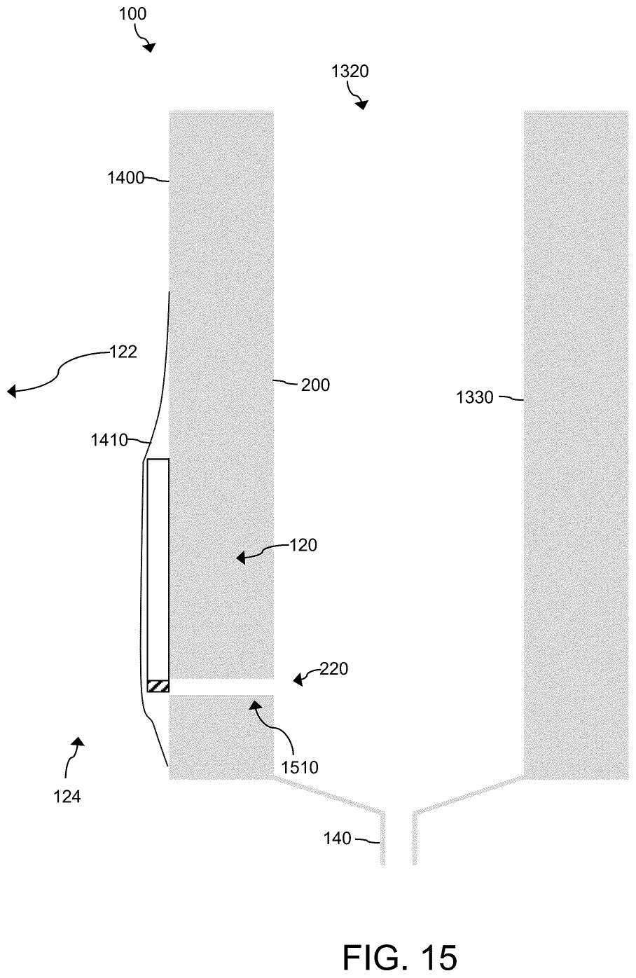

An exposure window 220 is defined between the internal wall 210 and the external wall 1400, as illustrated in FIG. 15 which is a cross section of the external wall 1400 through plane 1500 in FIG. 14. The exposure window 200 includes a channel 1510 that extends between the internal wall 210 and the external wall 1400. The channel 1510 is aligned with the base 122 and/or the sample pad 124 of the test strip 120. The liquid (e.g., liquid 130) can flow or pass through the channel 1310 and onto the base 122 and/or the sample pad 124 of the test strip 120 to allow the test strip 120 to automatically test the liquid for the presence of one or more substances. A second internal wall 1330 (e.g., the other internal side of the barrel 100) is shown in FIG. 13 to illustrate the internal volume/cavity 1320.

The covering material 1410 is attached to the external wall 1400. The covering material 1410 can be attached to the test strip 120 and/or can be tightly disposed around the test strip 120 as to restrict movement of the test strip 120 relative to the external wall 1400. The covering material 1410 can form a fluid seal with the external wall 1400 to prevent liquid (e.g., liquid 130) from escaping.

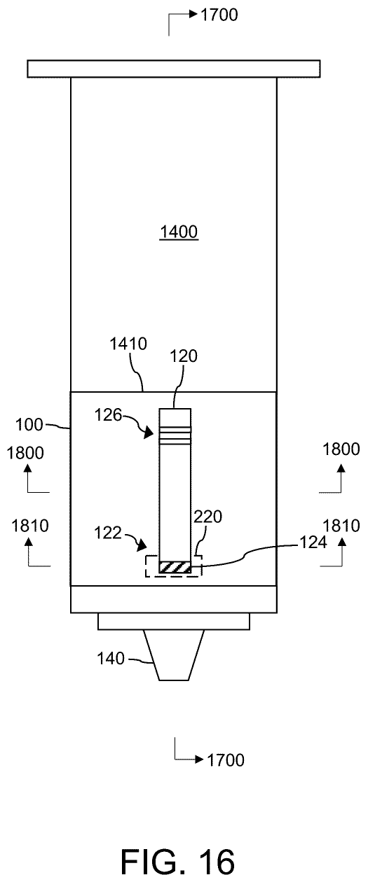

FIG. 16 is a side view of an external wall 1400 of the barrel 100, as seen from the outside of the barrel 100, according to another embodiment. The plunger 110 and liquid 130 are not illustrated in this figure for clarity purposes only. The embodiments illustrated in FIGS. 14 and 16 are the same except that in FIG. 14 the covering material 1410 is disposed on and covers only the test strip 120 and the external wall 1400 in close proximity to the test strip 120 while in FIG. 16 the covering material 1410 extends along some or all of the circumference of the external wall 1400.

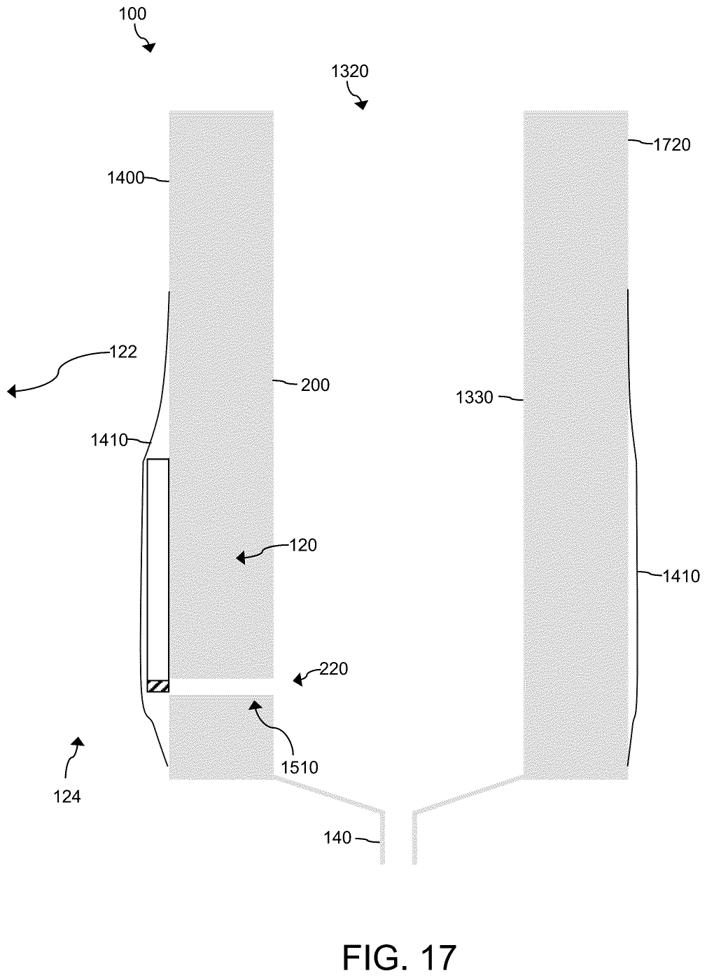

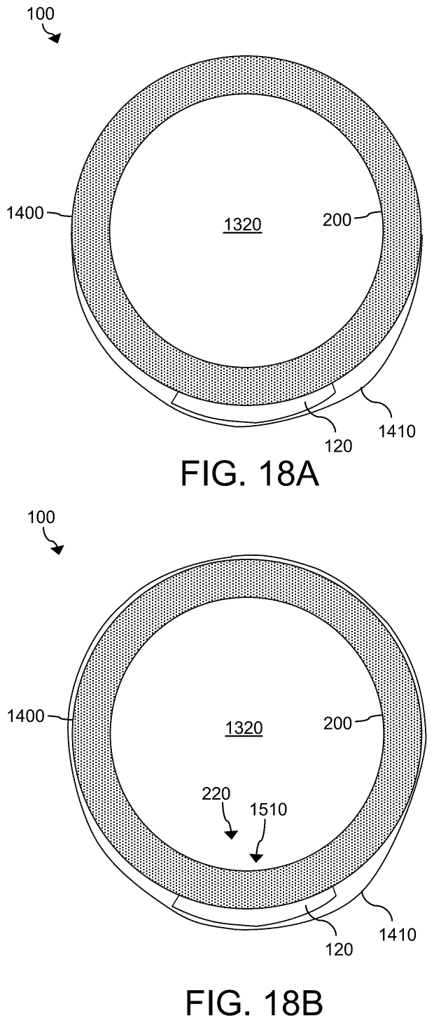

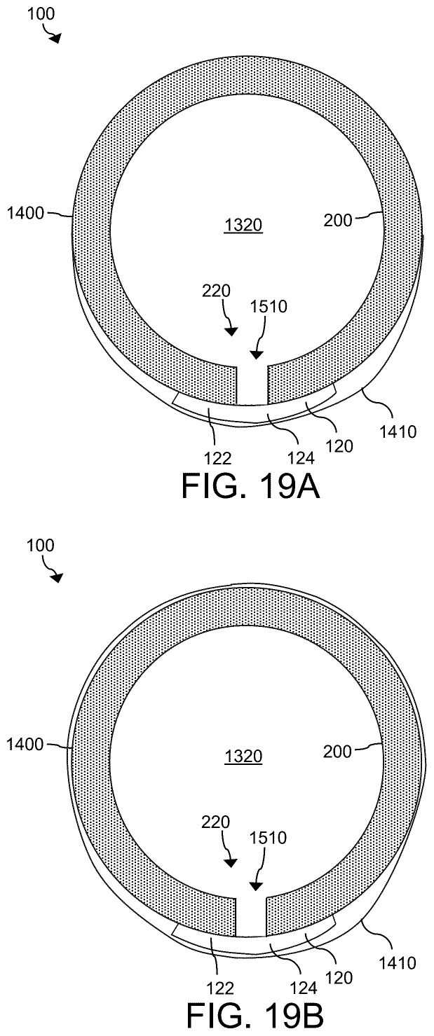

FIG. 17 is a cross section of the external wall 1400 through plane 1700 in FIG. 16 according to an embodiment. The cross sections shown in FIGS. 15 and 17 are the same except that in FIG. 17 the covering material 1410 extends to a second external wall 1720 (e.g., the other external side of the barrel 100). The covering material 1410 can extend partially along (e.g., along about half of) the circumference of the external wall 1400, for example as illustrated in FIGS. 18A and 18B. Alternatively, the covering material 1410 can extend fully along the circumference of the external wall 1400 so as to form a tube, hollow cylinder, or a ring, for example as illustrated in FIGS. 19A and 19B. FIGS. 18A and 18B are cross sections of the barrel 100 taken through plane 1800 (FIG. 17) according to different embodiments. FIGS. 19A and 19B are cross sections of the barrel 100 taken through plane 1810 (FIG. 17) according to different embodiments. FIGS. 18A and 19A can represent an implementation of FIGS. 16 and 17. FIGS. 18B and 19B can represent another implementation of FIGS. 16 and 17.

While example embodiments are illustrated, it is understood that the covering material 1410 can be disposed on and cover different percentages of the circumference of the external wall 1400 other than those illustrated. For example, the covering material 1410 can be disposed on and cover about 10% to about 100% of the circumference of the external wall 1400, including about 25%, about 50%, about 75%, and any range or value between any two of the foregoing percentages. The thickness of the covering material 1410 can be uniform or variable along the circumference of the external wall 1400. In some embodiments, the covering material 1410 can consist of or can comprise a shrink-wrap or a heat-shrink material.

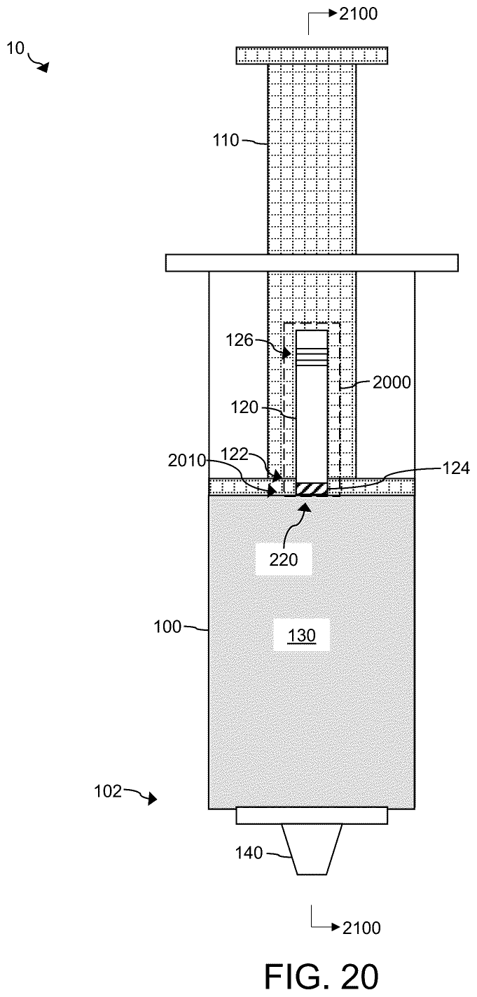

FIG. 20 is a side view of a syringe 10 according to another embodiment. The test strip 120 is disposed in a cavity 2000 defined in the plunger 110. An exposure window 220 is defined at the base 122 of the test strip 120 and/or at a base 2010 of the cavity 2000 to allow a liquid 130 to pass onto the base 122 to wet (e.g., make direct physical contact with) the sample pad 124.

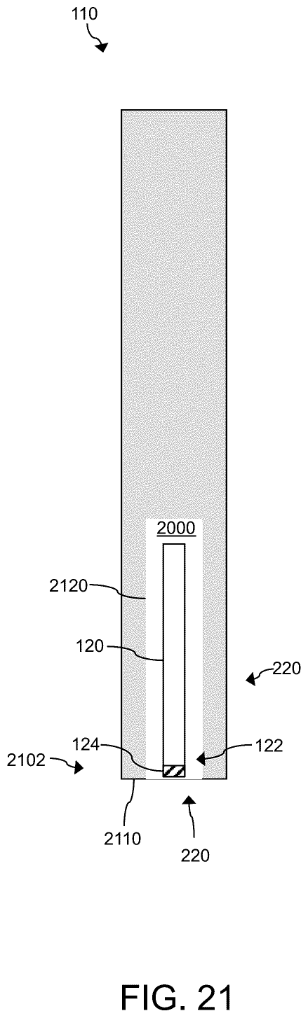

FIG. 21 is a cross section of the plunger 110 taken through plane 2100 in FIG. 20 according to an embodiment. In this cross section, the cavity 2000 extends to a distal end 2102 (e.g., to a base 2110) of the plunger 110 to form the exposure window 220. When the liquid 130 is disposed in the barrel 100, the liquid 130 flows into and contacts the base 122 of the test strip 120 (e.g., through the cavity 2000) including the sample pad 124 to automatically test the liquid 130 for one or more substances. The plunger 110 can be formed of an optically clear material that allows the test lines 126 (FIG. 20) to be viewed. The test strip 120 may extend any length of the plunger 110 to allow viewing of the results externally when the plunger 110 is minimally to maximally withdraw from the syringe barrel 100.

The cavity 2000 can be rectangular, round (e.g., circular), or another shape. The shape of the cavity 2000 can conform and/or can be complementary to the shape of the test strip 120.

The cavity 2000 can be in direct physical contact with the test strip 120. In some embodiments, a fluid seal can be formed between an internal wall 2120 of the plunger 110 and the test strip 120 form a fluid seal between the internal wall 2120 and the test strip 120. Alternatively, the cavity 2000 can be filled with a material, such as a covering material, that prevents the liquid from directly physically contacting a portion of the test strip 120 other than the base 122 and/or the sample pad of the test strip 120.

The invention should not be considered limited to the particular embodiments described above, but rather should be understood to cover all aspects of the invention as fairly set out in the attached claims. Various modifications, equivalent processes, as well as numerous structures to which the invention may be applicable, will be apparent to those skilled in the art to which the invention is directed upon review of this disclosure. The claims are intended to cover such modifications and equivalents.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

What is claimed is:

1. A syringe comprising:
a barrel;
a plunger configured to slidingly engage the barrel to receive or dispense a liquid; and
a test strip disposed on or in the barrel and configured to detect for a presence of one or more substances in the liquid.

2. The syringe of claim 1, wherein the one or more substances comprise an opioid.

3. The syringe of claim 2, wherein the opioid comprises fentanyl.

4. The syringe of claim 1, wherein the plunger is configured to crush the test strip when the plunger is moved towards a tip of the barrel.

5. The syringe of claim 1, further comprising a covering material attached to an internal wall of the barrel, the test strip disposed between the covering material and the internal wall, wherein:
a test window is defined in the covering material at a base of the test strip, and
only the base of the test strip is configured to be exposed to the liquid.

6. The syringe of claim 5, wherein a test region of the test strip faces the internal wall.

7. The syringe of claim 5, wherein the covering material comprises a tube.

8. The syringe of claim 5, wherein the covering material comprises a plastic and/or a polymer.

9. The syringe of claim 1, further comprising a covering material attached to an internal wall of the barrel, the test strip disposed between the covering material and the internal wall, wherein:
a channel is defined in the internal wall, the channel extending to a base of the test strip to form a test window, and
only the base of the test strip is configured to be exposed to the liquid.

10. The syringe of claim 1, wherein:
the test strip is disposed in a cavity defined in an internal wall of the barrel, and
a channel is defined between the cavity and an internal volume of the barrel, the channel aligned with a base of the test strip such that only the base of the test strip is configured to be exposed to the liquid.

11. A syringe comprising:
a barrel defining a test window;
a plunger configured to slidingly engage the barrel to receive or dispense a liquid; and
a test strip configured to detect for a presence of one or more substances in the liquid, the test strip disposed on an external wall of the barrel, the test strip having a base in fluid communication with the test window such that only the base of the test strip is configured to be exposed to the liquid.

12. The syringe of claim 11, wherein the test window includes a channel that extends from an internal wall to the external wall of the barrel.

13. The syringe of claim 11, further comprising a covering material attached to the external wall, the test strip disposed between the covering material and the external wall.

14. The syringe of claim 13, wherein the covering material comprises a shrink wrap.

15. The syringe of claim 13, wherein the covering material comprises a tube.

16. The syringe of claim 13, wherein the test strip is encased in the covering material.

17. A syringe comprising:
a barrel;
a plunger configured to slidingly engage the barrel to receive or dispense a liquid, the plunger defining a test window and a cavity; and
a test strip configured to detect for a presence of one or more substances in the liquid, the test strip disposed in the cavity, the test strip having a base in fluid communication with the test window such that only the base of the test strip is configured to be exposed to the liquid.

18. The syringe of claim 17, wherein the test window is defined in a base of the plunger.

19. The syringe of claim 17, wherein portions of the test strip other than the base are fluidly sealed in the cavity.
